(19) United States
(12) Patent Application Publication
(10) Pub. No.: US 2005/0169920 A1
(43) Pub. Date: Aug. 4, 2005

(54) Pecam-1 modulation

(76) Inventors: Jonathan M Gibbins, Reading (GB); Milenko Cicmil, Reading (GB)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/500,027

(22) PCT Filed: Dec. 17, 2002
(86) PCT No.: PCT/GB02/05730

(30) Foreign Application Priority Data
Dec. 22, 2001 (GB) 0130832.9

(51) Int. Cl. A61K 39/395; C07K 16/46

(52) U.S. Cl. 424/144.1; 530/388.22; 530/352

(57) ABSTRACT

Activation of PECAM-1 (platelet endothelial cell adhesion molecule-1) with, for example, a small molecule or antibody derivative provides a new therapeutic route for modifying platelet activation and thrombus formation which has utility in the treatment or prevention of cardiovascular diseases such as thrombosis, stroke, and vascular occlusion and in the treatment or prevention of homeostasis disorders. This treatment approach activates a general inhibitory mechanism. A screen for activators of PECAM-1 include PECAM-1, an ectodomain of PECAM-1, the cytoplasmic tail of PECAM-1, the ITIM of PECAM-1, an active site of PECAM-1, a recombinant extracellular domain of PECAM-1, or a part of derivative thereof and ways for detecting activation or cross-linking or phosphorylation or tyronsine phosphorylation of PECAM-1, an ectodomain of PECAM-1, the cytoplasmic tail of PECAM-1, the IPIM of PECAM-1, an active site of PECAM-1, a recombinant extracellular domain of PECAM-1, or a part or derivative thereof.

### FIELD OF THE INVENTION

The invention relates to the modulation of the activity of platelet endothelial cell adhesion molecule-1 (PECAM-1, CD31) for the treatment of or for reducing the occurrence of cardiovascular conditions such as thrombosis, vascular occlusion stroke and for the treatment of or for reducing the occurrence of haemostasis disorders.

### REVIEW OF THE ART KNOWN TO THE APPLICANT(S)

Thrombosis, an aggregation of blood factors primarily platelets and fibrin with entrapment of cellular elements frequently causing vascular obstruction at the point of its formation, is a remarkably prevalent problem. It underlies most deaths from cardiovascular disease (whether there is another underlying cause or not). The 2001 Heart and Stroke Statistical Update (American Hearth Association) reports the prevalence of cardiovascular disease to be 1 in 5 Americans with 12.4 million Americans currently suffering from coronary heart disease (myocardial infarction and angina pectoris) and 4.7 million suffer from the effects of stroke. In 1998 cardiovascular disease (which also includes the effects of high blood pressure and congestive heart failure) killed 915,619 people in America, corresponding to 1 in every 2.5 deaths.

Platelets initiate blood clotting and control the formation of clots and/or thrombi, rapid and complete activation of platelets at sites of tissue damage is ensured through numerous positive feedback pathways, mainly through the actions of mediators such as thromboxane A₂ and ADP that are released from activated platelets. The existence of such a rapid and reactive system emphasises the need for effective regulation of platelet function in order to prevent disorders such as thrombosis and haemorrhage. Platelet reactivity is a controlled balance between positive and negative regulatory factors and signalling mechanisms.

The use of reagents that target and block activatory platelet cell surface receptors is known, and has provided a successful approach in the development of anti-thrombotic drugs. However, due to the complex nature of the homeostasis system and the many ways it can be activated, none of these drugs has produced either a cure or a complete solution to all thrombosis related conditions. At best, in some trials, mortality has been reportedly reduced by up to 25%, however numerous side effects are associated with existing treatments. As a result almost all these drugs are used in combination depending on the clinical circumstances, which serves to highlight the need for a safe effective treatment for thrombosis and related cardiovascular conditions.

Much attention has recently been focussed on the identification of the receptors and signalling pathways that lead to platelet activation, particularly on exposure to collagen, thrombin and ADP.

Despite this intensive research activity the use of an agent which activates natural inhibitory receptors so as to modify, or inhibit platelet activation and thereby reduce thrombus formation has not previously been either suggested or attempted.

The inventors have identified a negative regulation system that is mediated via a cell-surface ITIM-bearing adhesion receptor, PECAM-1. PECAM-1 signalling results in a negative feedback on platelet activation pathways and thereby sets the threshold stimulation level for platelet activation in the absence of injury thus preventing thrombosis..

They have ascertained that activation of PECAM-1 with for example a small molecule or antibody derivative provides a new therapeutic route for modifying platelet activation and thrombus formation which has utility in the treatment or prevention of cardiovascular diseases, such as thrombosis, stroke and vascular occlusion and in the treatment or prevention of haemostasis disorders.

The present invention provides a far better approach to the treatment of these conditions than existing drugs as it activates a general inhibitory mechanism rather than merely blocking one arm of the activation system.

In its broadest aspect the invention provides a method of activating PECAM-1 for modifying or reducing or inhibiting platelet activation, or platelet aggregation, or platelet secretion.

In a preferred aspect the invention provides a method for activating or cross-linking or phosphorylating PECAM-1 for the treatment of or for reducing the occurrence of cardiovascular diseases such as thrombosis, vascular occlusion or stroke, or for the treatment of or for reducing the occurrence of haemostasis disorders.

In a further aspect the invention provides an activator for use in this method.

In a further aspect the invention provides an activator for the treatment of or for reducing the occurrence of cardiovascular diseases such as thrombosis, vascular occlusion or stroke, or for the treatment of or for reducing the occurrence of haemostasis disorders.

In another aspect the invention provides an activator for use in the manufacture of a medicament for the treatment of or for reducing the occurrence of cardiovascular diseases such as thrombosis, vascular occlusion or stroke, or for the treatment of or for reducing the occurrence of haemostasis disorders.

In another aspect the invention provides a screen for activators of PECAM-1 comprising PECAM-1, an ectodomain of PECAM-1, the cytoplasmic tail of PECAM-1, the ITIM of PECAM-1, an active site of PECAM-1, a recombinant extracellular domain of PECAM-1, or a part or derivative thereof and means for detecting activation or cross-linking or phosphorylation or tyrosine phosphorylation of PECAM-1, an ectodomain of PECAM-1, the cytoplasmic tail of PECAM-1, the IPIM of PECAM-1, an active site of PECAM-1, a recombinant extracellular domain of PECAM-1, or a part or derivative thereof.

### DESCRIPTION OF THE PREFERRED EMBODIMENT

The invention will now be described by way of example with reference to the accompanying experimental data and drawings in which:

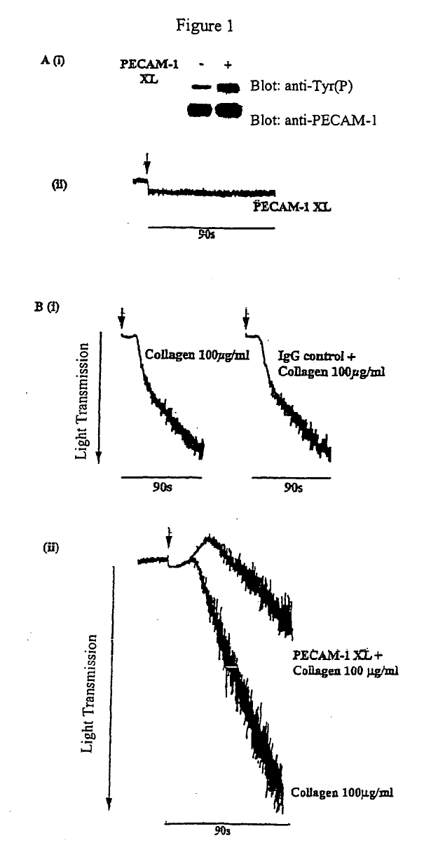

FIG. 1. Cross-linking of PECAM-1 inhibits collagen-stimulated platelet aggregation. (A) PECAM-1 cross-linking on platelet surfaces results in its tyrosine phosphorylation and does not stimulate platelet aggregation. (i) PECAM-1 was immunoprecipitated from washed human platelets under resting conditions or following PECAM-1 cross-linking. Proteins were separated by SDS-PAGE and immunoblotted to detect phosphotyrosine residues (upper panel). Immunoprecipitation was verified by reprobing for PECAM-1 (lower panel). (ii)

PECAM-1 was cross-linked on washed platelets and aggregation was monitored using optical aggregometry. (B) (i) Platelets were incubated with isotype-matched control IgG for 5 minutes before the addition of F(ab′)₂ cross-linker for 90 seconds and then were stimulated with collagen (100 mM (mg/mL)). Aggregation was monitored using optical aggregometry. (ii) Cross-linking of PECAM-1 inhibits collagen-stimulated platelet aggregation. Platelets were stimulated with collagen (100 mM (mg/mL)) for 90 seconds with and without first cross-linking PECAM-1 and platelet aggregation monitored by optical aggregometry. Data are representative of 3 separate experiments. Tyr(P), tyrosine phosphorylation; PECAM-1 XL, PECAM-1 cross-linking.

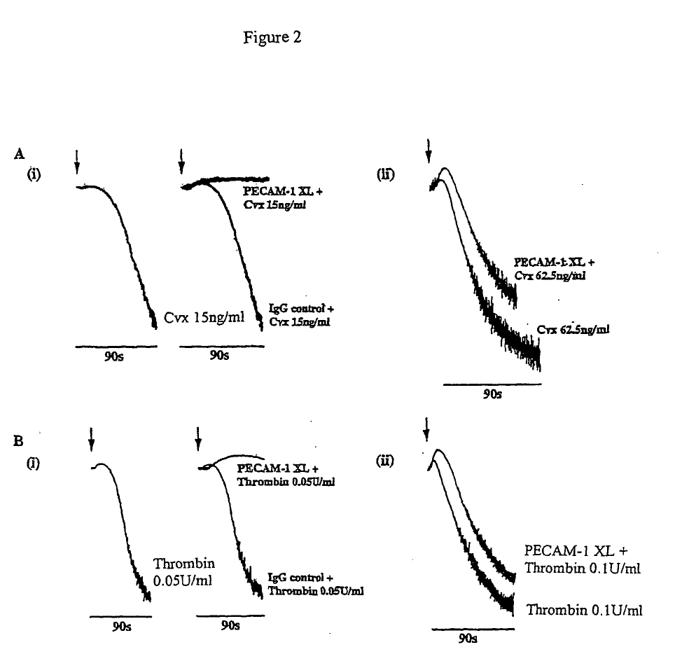

FIG. 2. PECAM-1 cross-linking inhibits GPVI- and thrombin receptor-stimulated platelet aggregation. (A) Platelets were stimulated with Cvx at (i) 15 nM (ng/mL) and (ii) 62.5 nM (ng/mL) with and without first cross-linking PECAM-1, and aggregation responses were monitored by optical aggregation (arrow indicates the addition of Cvx). (B) Platelets were stimulated with thrombin at (i) 0.05 U/mL and (ii) 0.1 U/mL with and without prior cross-linking of PECAM-1. Platelet aggregation was monitored by optical aggregometry (arrow indicates the addition of thrombin). Treatment of platelets with isotype-matched control IgG before stimulation with Cvx (15 nM (ng/mL)) and thrombin (0.05 U/mL) is shown in A(i) and B(i), respectively. Data are representative of 3 separate experiments. PECAM-1 XL, PECAM-1 cross-linking.

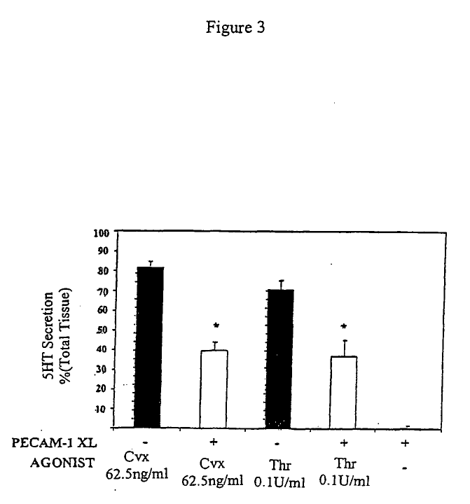

FIG. 3. Platelet-dense granule secretion is inhibited by PECAM-1 signalling. Platelets were loaded with [³H]5-HT before stimulation with Cvx (62.5 nM [ng/mL]) or thrombin (0.1 U/mL). Where required, PECAM-1 was cross-linked prior to the addition of an agonist. Secretion of [³H]5-HT into cell medium was measured using scintillation spectrometry. [³H]5-HT release is ex-pressed as a percentage of total tissue content after subtraction of basal secretion values. Results represent mean +/− SE (n=3). Student's t-test was used to compare PECAM-1 cross-linked and non-cross-linked sample for statistical significance. (* indicates p-values <0.05. PECAM-1 XL, PECAM-1 cross-linking

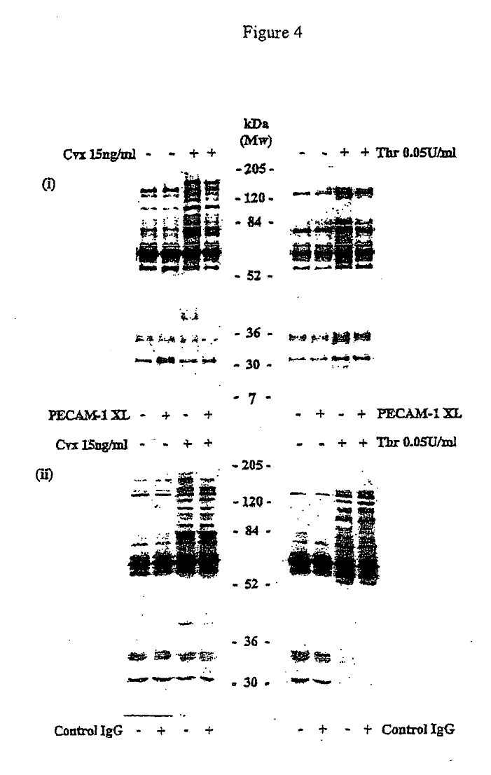

FIG. 4. PECAM-1 signaling inhibits platelet protein tyrosine phosphorylation. Platelet lysates were prepared in Laemmli buffer from nonstimulated platelets (stirred with buffer alone) and platelets stimulated for 90 seconds with Cvx (15 nM [ng/mL]) or thrombin (0.05 U/mL). Before stimulation, PECAM-1 was cross-linked in some samples (A) or platelets were incubated with isotype-matched control IgG and cross-linker F(ab′)₂ (B). Proteins were separated by SDS-PAGE under reducing conditions and immunoblotted to detect protein tyrosine phosphorylation. PECAM-1 XL, PECAM-1 cross-linking

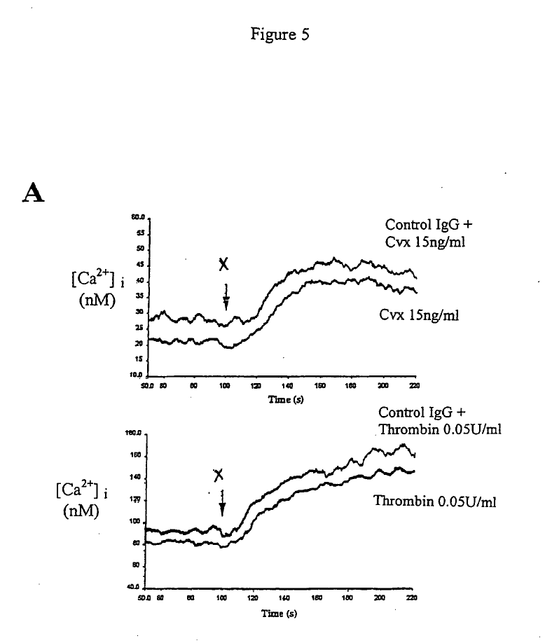

FIG. 5. PECAM-1 cross-linking inhibits the mobilization of calcium from intracellular stores. Fura-2 AM loaded human platelets were stimulated with either Cvx or thrombin, and the mobilization of calcium was measured fluorometrically (arrow indicates the addition of agonist). Panel A) Treatment of platelets with control IgG and cross-linker F(ab′)₂ has no effect on Cvx- and thrombin-stimulated calcium mobilization.

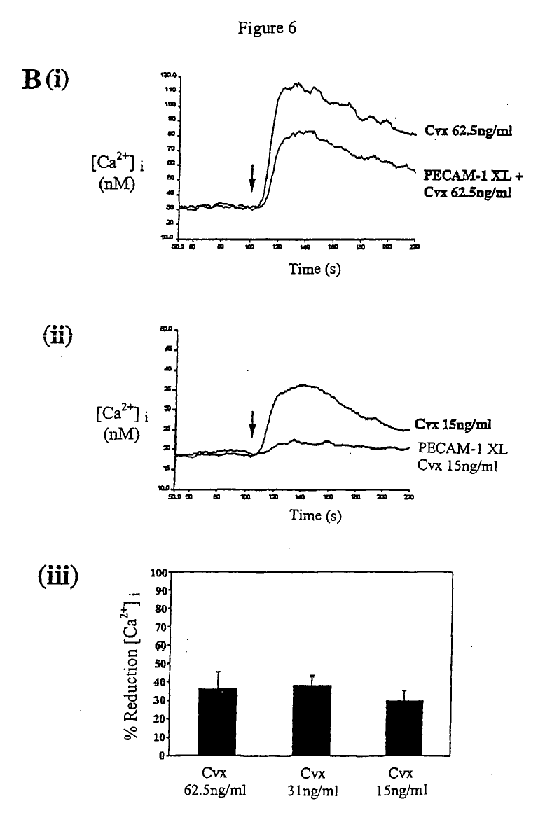

FIG. 6: Panels B i and ii) show representative calcium responses for stimulation with Cvx at 62.5 nM (ng/mL) and 15 nM (ng/mL), respectively, with and without first cross-linking PECAM-1. Traces are representative of 3 separate experiments. Panel (iii) shows PECAM-1-induced percentage reduction in peak levels of intracellular calcium stimulated by 62.5, 31.25, and 15 nM (ng/mL) Cvx. Data presented represents mean +/− SE (n=3).

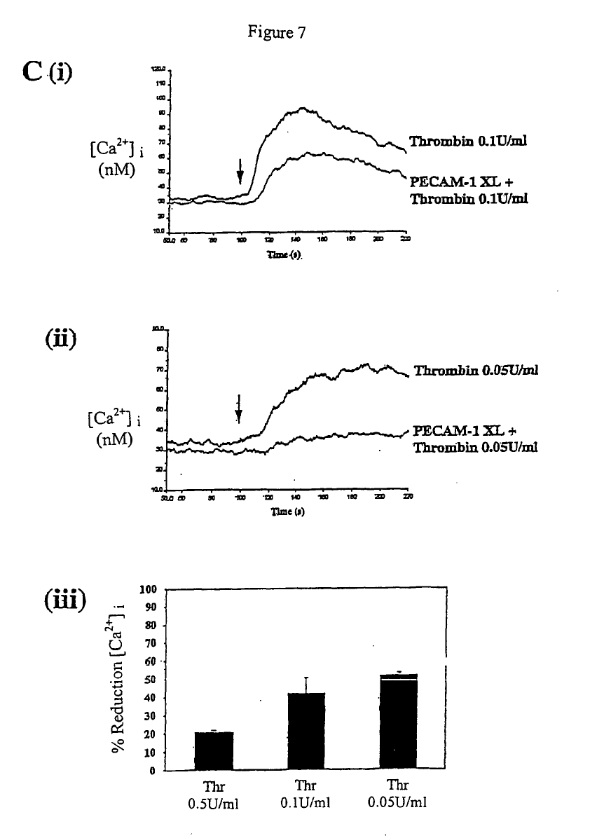

FIG. 7: Panels C i, and ii) show representative calcium responses for stimulation with thrombin at 0.1 U/mL and 0.05 U/mL, respectively, with and without first cross-linking PECAM-1. Traces are representative of 3 separate experiments. Panel (iii) PECAM-1-induced percentage reduction in peak levels of intracellular calcium stimulated by 0.5, 0.1, and 0.05 U/mL thrombin. Data represent mean +/− SE (n=3)). PECAM-1 XL, PECAM-1 cross-linking.

Platelet endothelial cell adhesion moleule-1 PECAM-1 is a 130 kDa membrane-spanning glycoprotein whose expression is restricted to several haematopoietic cell types including platelets, monocytes, neutrophils, certain T-lymphocytes and also vascular endothelial cells. The functions of PECAM-1 are diverse and include angiogenesis, vasculogenesis, integrin regulation, transendothelial migration of leukocytes, and T- and B-cell antigen receptor function, although the role of this molecule in platelets is presently unclear. When PECAM-1 was cloned it was assigned to the family of cell adhesion molecules on the basis of structural similarities. PECAM-1 is involved in adhesion although much attention has been recently been directed to studying its ability to participate in signal transduction. The cytoplasmic tail of PECAM-1 contains a conserved motif called an immunoreceptor tyrosine based inhibitory motif (ITIM), which underlies its signaling properties and is shared by a growing family of inhibitory receptors. These include the immunoglobulin G receptor FcγRIIB, the killer inhibitory receptors (KIR) and signal regulatory proteins (SIRPs), although PECAM-1 is the only ITIM-bearing receptor that has been reported to be expressed in platelets. It has therefore been proposed that PECAM-1 should be assigned to the Ig-ITIM family of receptors.

The ligand binding properties of PECAM-1 are complex. It has the capacity for homophilic interactions, and also heterophilic interactions with a number of molecules that include integrin αᵥβ₃ and CD38, PECAM-1 becomes phosphorylated on tyrosine residues in response to a variety of stimuli that include PECAM-1 cross-linking, activation of the high affinity receptor for immunoglobulin E (FcεRI), shear and oxidative stress. The inventors have recently found that platelet activation via the collagen receptor glyoprotein GPVI (GPVI) and thrombin receptors result in PECAM-1 tyrosine phosphorylation which is not dependent on platelet aggregation and secretion, although tyrosine phosphorylation is enhanced by aggregation. The tyrosine residues that become phosphorylated in PECAM-1 have been mapped and fall within the ITIM. Phosphorylated ITIMs recruit signalling molecules such as the tyrosine phosphatases SHP-1 and SHP-2 that bind to the motif via Src-homology 2 domain interactions. Indeed, both SHP-1 and SHP-2 have been shown to associate with tyrosine phosphorylated PECAM-1, and PECAM-1 ITIM phosphopeptides activate these phosphatases in vitro. Generally, these protein tyrosine phosphatases exhibit inhibitory effects by counteracting tyrosine kinase-dependent pathways, although SHP-2 has been shown to positively regulate growth factor receptor signalling.

Immunoreceptor tyrosine-based activatory motif-(ITAM) bearing receptors have been shown to have a critical place in the regulation of platelet function. Indeed the collagen receptor GPVI-FcR γ-chain complex signals through an ITAM present on the cytoplasmic tail of the FcR γ-chain. Several studies in other cell systems have provided evidence of an antagonistic relationship between ITAM and ITIM containing receptors when expressed on the same cell. An example of this is the receptor for IgG FcγRIIB (ITIM) that negatively regulates cell activation stimulated by FcγRIIA (ITAM).

PECAM-1 was stimulated through cross-linking using antibodies directed to the extracellular domain of the receptor. This strategy was the chosen as the most specific manner to activate PECAM-1, and activation was confirmed since cross-linking stimulated its tyrosine phosphorylation. PECAM-1 cross-linking stimulates tyrosine phosphorylation (FIG. 1) and association of SHP-2 (not shown) but does not itself cause platelet activation. Tyrosine phosphorylation of PECAM-1 on cross-linking was shown not to be dependent on integrin α_{iib}β₃ engagement. Cross-linking PECAM-1 for 90 s prior to stimulation with collagen caused inhibition of platelet aggregation. At lower concentrations of collagen, aggregation was inhibited completely, but even at very high concentrations of collagen (100 μg/ml) PECAM-1 activation caused a substantial inhibition of aggregation. Since collagen is also able to bind other receptors on the platelet, including the integrin α₂β₁, the effect of PECAM-1 cross-linking on GPVI-mediated platelet aggregation using the specific agonist Cvx was examined. Similar results were obtained, with complete inhibition of aggregation at lower concentrations of agonist (15 ng/ml) and partial effects at higher concentrations (62.5 ng/ml), indicating that PECAM-1 is a potent inhibitor of GPVI-mediated (ITAM) platelet activation.

Experiments were coducted to determine whether the inhibitory effect of PECAM-1 is restricted to signalling via ITAM containing receptors. On the contrary, PECAM-1 cross-linking was also found to inhibit thrombin-stimulated platelet aggregation. Although no inhibitory effect of PECAM-1 was observed at moderate concentrations of thrombin (0.5 and 1 U/ml, results now shown) dramatic levels of inhibition were observed at very high concentrations of collagen (100 μg/ml). PECAM-1 mediated inhibition of thrombin-stimulated aggregation was observed only a lower thrombin concentrations (complete inhibition with 0.05 U/ml and slight inhibition at 0.1 U/ml). Showing that PECAM-1 activation inhibits thrombin-stimulated platelet aggregation less efficiently than collagen-stimulated aggregation.

The effect of PECAM-1 cross-linking on platelet aggregation stimulated by other G protein-coupled receptor agonists was also examined. Aggregation in response to low concentrations of the thromboxane mimetic U46619 were also reduced by PECAM-1 signalling. In addition, preliminary work suggests that ADP-induced platelet aggregation at low agonist concentrations may also be affected.

The inventors have demonstrated that activation of PECAM-1 signalling by antibody-mediated cross-linking results in inhibition of collagen-mediated activation. They have also shown that the effects of the GPVI-selective agonist convulxin, are inhibited by activation of PECAM-1 signalling. Thrombin-stimulated activation has also been shown to be inhibited by the activation of PECAM-1 signalling, indicating that the inhibitory effects of PECAM-1 are not restricted to the inhibition of ITAM-containing receptor signalling pathways.

This is consistent with PECAM-1 performing a negative regulatory role in the control of platelet activation stimulated by both ITAM- and non-ITAM-containing receptor agonists.

Even though previous studies by for example Duncan G S, Andrew D P, Takimoto H, et al: Genetic evidence for functional redundancy of platelet/endothelial cell adhesion molecule-1 (PECAM-1): CD31-deficient mice reveal PECAM-1-dependent and PECAM-1-independent functions. J. Immun. 1999; 162:3022-3030, and Patil S, Newman D K, Newman P J: PECAM-1 serves as an inhibitory receptor that modulates platelet responses to collagen. Blood. 2001; 97:1727-1732, have reported that thrombin and ADP stimulated platelet aggregation in PECAM-1 -deficient mouse platelets is normal.

The observation that ITAM-mediated signalling is inhibited to a higher degree than non-ITAM-mediated signalling is further supported by experiments in platelets where PECAM-1 was co-ligated with FcγRIIA (an ITAM-containing immunoglobulin G receptor). Co-ligation results in inhibition of FcγRIIA-mediated platelet aggregation and intracellular calcium mobilisation stimulated by receptor cross-linking.

The inhibitory effects of PECAM-1 on stimulation with agonists such as the collagen, Cvx, thrombin, and the thromboxane mimetic U46619, suggest that PECAM-1 inhibits primary signalling events and also secondary stimulation by factors released by activated platelets. It is possible, therefore, that inhibition of receptors that cross-talk with GPVI-mediated signalling may contribute to the inhibitory actions of PECAM-1 on collagen- and convulxin-stimulated activation. This may also explain why the inhibitory functions of PECAM-1 on GPVI-mediated activation are more effective than on thrombin-mediated activation.

Having established that PECAM-1 cross-linking inhibits platelet function, the effect of this on some aspects of signal transduction was examined. Platelet activation was found to be accompanied by a concomitant inhibition of platelet protein tyrosine phosphorylation and decreased levels of calcium mobilisation from intracellular stores. Platelet activation by the collagen receptor GPVI is dependent on tyrosine kinases, and consequently is associated with the rapid tyrosine phosphorylation of a wide variety of platelet proteins. Stimulation with thrombin results in protein tyrosine phosphorylation, but to a lesser degree. Cross-linking PECAM-1, which in itself does not alter protein tyrosine phosphorylation levels, inhibits substantially the level of tyrosine phosphorylation that is induced by subsequent stimulation with Cvx or thrombin. This is consistent with the reduction in aggregation and secretion observed. The identities of the phosphoproteins whose phosphorylation is reduced on PECAM-1 cross-linking is currently under investigation.

Underlying the PECAM-1 mediated inhibition of platelet activation, is a significant level of inhibition of calcium release from intracellular stores. As seen with aggregation assays, at lower concentrations of Cvx and thrombin (15 ng/ml) ad 0.05 U/ml, respectively) PECAM-1 signalling inhibits release almost completely, where a partial effect is observed at higher agonist concentrations. Calcium mobilisation is stimulated through the intracellular generation of inositol 1,4,5-trisphosphate (IP₃) from phosphatidylinositol 4,5-bisphosphate by phospholipase C. It is well established that in platelets, stimulation with collagen leads to phosphorylation and activation of PLCγ2 isoform, and thrombin signalling regulates PLC_{β}. PECAM-1 cross-linking results in a substantial and significant reduction in total inositol phosphates stimulated by Cvx and thrombin. Which suggests that PECAM-1 exerts its effects on collagen- and thrombin-stimulated signalling either upstream of the PLC isoforms, or on these enzymes themselves.

Methods

Materials—Horm-Chemie collagen (collagen-fibres from equine tendons) was purchased from Nycomed (Munich, Germany). Convulxin was purified from the venom of the rattlesnake (Crotalus durissus terrificus) as described previously. The thromboxane mimetic U46619 and ADP was purchased from Sigma (Poole, UK). Anti-phosphotyrosine monoclonal antibody (4G10) was from Upstate Biotechnology (TCS Biologicals, Buckinghamshire, UK). Anti-PECAM-1 antibodies: monoclonal antibody HCl/6 was from Serotec (Oxford, UK); polyclonal anti-PECAM-1 (C-20) and monoclonal antibody AB468 were from Autogen Bioclear Ltd. (Wiltshire, UK); monoclonal antibody PECAM 1.3 was kindly provided by Professor Peter Newman (The Blood Centre of Woutheastem Wisconsin, Milwaukee, Wis., USA). Control mouse IgG, was purchased from Sigma (Poole, UK). Monoclonal antibody IV.3 was purified from hybridoma cell culture medium and F(ab′)₂ fragments generated by pepsin digestion using reagents purchased from Pierce (Perbio Scientific, Chester, UK). Horseradish peroxidase-conjugated secondary antibodies and the enhanced chemiluminescence detection system were purchased from Amersham Pharmacia Biotech (Buckinghamshire, UK). Fura-2 AM was from Molecular Probes (Cambridge Bioscience, Cambridge, UK).

Preparation and stimulation of Platelets: Human platelets from drug-free volunteers were prepared on the day of the experiment by differential centrifugation and suspended in modified Tyrodes-Hepes buffer (134 mM NaCl, 0.34 mM Na₂HPO₄, 2.9 mM KCl, 12 mM NaHCO₃, 20 mM Hepes, 5 mM glucose, 1 mM MgCl₂, pH7.3) to a density of 2×10⁸ cells/ml. For protein precipitation experiments, platelets were resuspended at 8×10⁸ cells/ml in buffer containing 1 mM EGTA to prevent aggregation. Stimulation of platelets (450 μl) with coliagen, convulxin (Cvx) and thrombin (delivered in 50 μl) was performed at 37° C. in an optical platelet aggregometer (Chrono-log Corporation) with continuous stirring (1200 rpm). PECAM-1 activation was stimulated by incubation with anti-PECAM-1 antibodies (AB468, 1 μ/ml or PECAM 1.3, 10 μg/ml) for 5 minutes, followed by incubation for 90 s with F(ab′)₂ fragments of anti-IgG secondary antibodies (30 μg/ml) to cross-link. mAb AB468 was generated against full length PECAM-1 ectodomain. Control experiments were performed using an irrelevant isotype-matched antibody in place of AB468 or PECAM-1.3 and used at the same concentration. In some experiments the low affinity receptor for IgG was blocked by incubation with a saturating concentration (1 μg/ml) of F(ab′)₂ fragments of mAbIV.3 for 5 minutes. Saturating concentrations of mAbIV.3 antibody were established by determining the concentration of F(ab′)₂ fragment that completely inhibited subsequent whole IgG-mediated FcγRIIA cross-linking and platelet activation. Platelet aggregation was determined by optical aggregometry.

Immunoprecipitation studies: Platelet stimulation was terminated by the addition of an equal volume of ice cold lysis buffer (2% [v/v] Nonidet P40, 20 mM Tris, 300 mM NaCl, 10 mM EDTA, 1 mM phenylmethylsulfonyl fluoride, 2 mM Na₃VO₄, 10 μg/ml leupeptin, 10 μg/ml aprotinin, 1 μg/ml pepstatin A, pH7.3). Detergent-insoluble debris was removed and the lysates were pre-cleared by mnixing with protein A-Sepharose for 1 hour at 4° C. (20 μl of a 50% [w/v] suspension of protein A-Sepharose of Tris-buffered saline-Tween [TBS-T: 20 mM Tris, 137 mM NaCl, 0.1% [v/v] Tween 20, pH7.6). Protein A-Sepharose was removed from the lysates before the addition of anti-PECAM-1 antibody (HCl/6, 1 μg). Following rotation at 4° C. for 1 hour, 0.5 μl secondary antiserum was added (rabbit anti-mouse IgG) and mixed for a further 30 min. 25 μl protein A-Sepharose suspension was added to each sample and mixing continued for 1 hour before washing the Sepharose pellet in lysis buffer followed by a wash with TBS-T, and the addition of Laemmli sample-treatment buffer. Proteins were separated by SDS-PAGE under reducing conditions using 10% gels and transferred to polyvinylidene difluoride membranes by semi-dry western blotting.

Immunoblotting: Membranes were blocked by incubation in 10% (w/v) bovine serum albumin (BSA) dissolved din TBS-T. Primary and secondary antibodies were diluted in TBS-T containing 2% (w/v) BSA, and incubated with membranes for 1 hour at room temperature. Blots were washed for 2 hours in TBS-T following each incubation with antibodies for 1 hour at room temperature, and then developed using an enhanced chemiluminescence detection system. Primary antibodies were used at a concentration of 1 μg/ml (anti-phosphotyrosine, 4G10; anti-PECAM-1, C-20) and horseradish peroxidase-conjugated secondary antibodies were diluted 1:10000.

5-Hydroxytryptamine (5-HT) secretion assay: Platelets were loaded with [³H]5-HT by incubation with 0.5 μCi/ml of platelet-rich plasma for 1 h at 37° C. Platelets were prepared from the platelet-rich plasma as described above. Stimulation of platelets was terminated by addition of an equal volume of 6% glutaraldehyde and microcentrifugation, and the level of [³H]5-HT release into the supernatant was determined by scintillation spectrometry. [³H]5-HT release was expressed as a percentage of the total tissue content following subtraction of release under basal conditions.

Measurement of [Ca²⁺]₁ by spectrofluorimetry: Washed human platelets (prepared as above) were incubated at 2×10⁹ cells/ml in calcium free Tyrodes-hepes buffer with 3 μM Fura-2 AM for 45 min. Platelets were washed once and resuspended at 2×10⁸ cells/ml in modified Tyrodes-hepes buffer. Stimulation of platelets (450 μl) in the presence of 2 mM EGTA with Cvx and thrombin (delivered in 50 μl) was performed with constant stirring at 37° C. in a luminescence spectrophotometer (LS-50B, Perkin Elmer) with excitation wavelengths of 340 nm and 380 nm. Fluorescence emission was measured at a wavelength of 510 nm. Where required, PECAM-1 was cross-linked prior to stimulation with agonist as described above. The ration of emission values (excitation at 340/380 nm) was calculated and converted to calcium concentration using FLWinLab software (Perkin Elmer) utilising the equation [Ca²⁺]₁ =K_{d}×(R−Rₘᵢₙ)/Rₘₐₓ−R)×SFB (where R is emission ratio value (340/380 nm). Rₘₐₓ the maximum 340/380 ratio, was determined by lysing platelets with 25 μM digitonin in the presence of 1 mM CaCl₂. The Rₘᵢₙ 340/380 ratio was obtained by addition of 2 mM EGTA. K_{d} is the dissociation constant of the Fura-2/Ca²⁺ complex (224 nM) and SFB is the fluorescence ratio at 340/380 nm of Rₘᵢₙ and Rₘₐₓ.

Statistical Analysis: Determination of statistical significance was performed using Student's paired T-test. The results are expressed as the mean +/− standard error of the mean (S.E.M.).

Experimental Data

Cross-linking PECAM-1 inhibits collagen-stimulated platelet aggregation: An antagonistic relationship has been reported between ITIM- and ITAM-containing receptors when expressed in the same cell. Since the platelet collagen receptor GPV1 signals through an ITAM present on the FcR γ-chain with which it is associated, the inventors investigated the effect of PECAM-1 signalling on platelet activation with collagen. PECAM-1 was activated by incubation with antibodies specific for the ectodomain of PECAM-1 (AB468 [FIG. 1] or PECAM-1.3 [not shown]) and cross-linked with a secondary antibody [F(ab′)₂ fragment]. This resulted in increased tyrosine phosphorylation of the protein, and did not result in stimulation of platelet aggregation (FIG. 1A). Tyrosine phosphorylation of PECAM-1 was maintained on cross-linking in the presence of EGTA (1 mM), RGDS peptide (0.5 mM) and the γ-chain peptide of fibrinogen (100 μM) added separately or altogether (not shown). This, together with the fact that these experiments were performed on washed platelets indicates that the tyrosine phosphorylation of PECAM-1 on cross-linking is not dependent on integrin α_{IIb}β₃ engagement. The effect of PECAM-1 cross-linking for 90 s prior to stimulation with collagen was found to have a marked inhibitory effect on collagen-stimulated platelet aggregation. At lower concentrations of collagen (e.g. 10 μg/ml), cross-linking of PECAM-1 before agonist addition completely abolished aggregation (data not shown). FIG. 1B(ii) shows the marked inhibitory effect of PECAM-1 cross-linking on a very high concentration of collagen (19 μg/ml). The use of an isotype-matched IgG control and cross-linker F(ab′)₂, had no effect of PECAM-1 tyrosine phosphorylation (not shown) and collagen-stimulated platelet aggregation [FIG. 1B(i)]. Results are representative of 3 separate experiments. Similar results were obtained using the alternative anti-PECAM-1 antibody PECAM 1.3. In some experiments the low affinity receptor for IgG FcγRIIA was blocked prior to PECAM-1 cross-linking and agonist stimulation using a saturating concentration of F(ab′)₂ fragments of mAbIV.3. The inhibitory effect of PECAM-1 cross-linking was unaltered under these conditions. This clearly indicates that the inhibitory effect of PECAM-1 using antibodies is not due to activation of FcγRIIA.

PECAM-1 cross-linking inhibits GPVI- and thrombin receptor-mediated platelet aggregation: Given the marked effect of PECAM-1 signalling on collagen-mediated platelet aggregation the inventors investigated whether this effect was restricted to GPVI-mediated signalling only. GPVI was stimulated using the selective agonist Cvx, a protein purified from the venom of the rattlesnake Crotalis durissus terrificus. Aggregation stimulated with 15 ng/ml Cvx was completely inhibited by prior activation of PECAM-1 [FIG. 2A(i)], and a partial inhibitory effect was observed at higher concentrations of Cvx [31.25 and 62.5 ng/ml, not shown and in FIG. 2A(ii), respectively]. Similar results were observed on stimulation of platelets with the G protein coupled receptor agonist thrombin. Complete inhibition of aggregation at 90 s stimulation was observed at a thrombin concentration of 0.05 U/ml [FIG. 2B(i) and a partial effect at 0.1 U/ml FIG. 2B(ii)]. The use of an isotype-matched IgG control and cross-linker F(ab′)₂ had no effect on Cvx- or thrombin-stimulated platelet aggregation [FIG. 2A(i) and 2B(i)]. No inhibitory effect of PECAM-1 activation was observed at higher concentrations of thrombin (e.g. 0.5 and 1 U/ml, data not shown). Results are representative of 5 separate experiments. Similar results were obtained using the alternative anti-PECAM-1 antibody PECAM-1.3, and when FcγRIIA was blocked prior to PECAM-1 cross-linking.

Platelet secretion is inhibited by PECAM-1 signalling: Platelet activation is accompanied by secretion from dense-granules. Dense granule secretion was assessed by measuring the release of [³H]5-HT from pre-loaded washed platelets. FIG. 3 shows the results of experiments to determine the effect of PECAM-1 cross-linking on [³H]5-HT secretion. A significant reduction in secretion was observed in platelets where PECAM-1 was activated before stimulation with Cvx (81.9% +/−2.9 to 39.8% +/−4.1, p=0.02, n=3) or thrombin (70.9% +/−4.6 to 37.0% +/−8.0, p=0.01, n=3). The use of an isotype-matched IgG control and cross-linker F(ab′)₂ had no effect on Cvx- or thombin-stimulated dense granule secretion (not shown). Experiments performed in the presence of mAb IV.3 to block the Fc receptor FcγRIIA produced similar results.

PECAM-1 inhibits platelet protein tyrosine phosphorylation: The effect of PECAM-1 cross-linking on GPVI- and thrombin receptor-stimulated signalling was also investigated. Platelets were stimulated with Cvx (15 ng/ml) or thrombin (0.05 U/ml) with or without prior cross-linking of PECAM-1 for 90 s. Whole cell protein tyrosine phosphorylation levels were determined by immunoblotting. Cross-linking PECAM-1 alone had no effect on basal platelet tyrosine phosphorylation levels (FIG. 4). Stimulation with Cvx (15 ng/ml) or thrombin (0.05 U/ml) caused an increase in the level of tyrosine phosphorylation of a broad range of proteins. In those samples where PECAM-1 signalling was stimulated by cross-linking before incubation with Cvx or thrombin, total tyrosine phosphorylation was reduced (FIG. 4). The use of an isotype-matched IgG control and cross-linker F(ab′)₂ had no detectable effect on Cvx- or thrombin-stimulated total tyrosine phosphorylation levels.

PECAM-1 inhibits the mobilisation of calcium from intracellular stores: Stimulation of the collagen receptor GPVI and thrombin receptors leads to rapid intracellular mobilisation of calcium, an effect that is essential for secretion and aggregation. Intracellular calcium levels were measured fluorometrically using the calcium sensitive dye Fura-2 AM. Experiments were performed in the presence of 2 mM EGTA to prevent the entry of extracellular calcium. Stimulation of platelets with Cvx and thrombin resulted in a rapid increase in the levels of intracellular calcium that declined over a period of approximately 5 minutes. The incubation of platelets with control antibody ad cross-linker F(ab′)₂ caused no change in Cvx- and thrombin-stimulated intracellular calcium mobilisation (FIG. 5). The cross-linking of PECAM-1 for 90 s before incubation with Cvx (62.5 ng.ml) or thrombin (0.1 U/ml) resulted in a markedly reduced level of calcium mobilisation [FIGS. 6B(i) and 7C(i)]. At the lower concentrations of agonists used (Cvx, 15 ng/ml; thrombin, 0.05 U/ml) calcium mobilisation was almost abolished [FIGS. 6B(ii) and 7C(ii)]. The effect of PECAM-1 cross-linking on the reduction of peak intracellular calcium levels for a range of agonist concentrations is shown in FIGS. 6B(iii) and 7C(iii). A reduction of at least 50% in calcium mobilisation was observed at all of the concentrations of Cvx and thrombin tested. Similar results were obtained using the alternative anti-PECAM-1 antibody PECAM-1.3. Furthermore, experiments performed in the absence of extracellular EGTA indicate that PECAM-1 cross-linking does not inhibit agonist-induced influx of calcium (data not shown).

1-18. (canceled)

19. The activation of PECAM-1 for modifying or reducing or inhibiting platelet activation, or platelet aggregation, or platelet segregation.

20. The activation claimed in claim 19 wherein the activation comprises cross-linking PECAM-1.

21. The activation claimed in claim 19 wherein the activation comprises antibody mediated cross-linking.

22. The activation claimed in claim 21 wherein the antibody is specific for the ectodomain of PECAM-1.

23. The activation claimed in claim 21 further comprising a secondary antibody.

24. The activation claimed in claim 19 wherein the activation comprises phosphorylation of PECAM-1.

25. The activation claimed in claim 24 wherein the phosphorylation occurs at the cytoplasmic tail of PECAM-1.

26. The activation claimed in claim 24 wherein the phosphorylation occurs within the ITIM of PECAM-1.

27. The activation claimed in claim 24 wherein PECAM-1 is phosphorylated at tyrosine residues.

28. The activation of cross-linking or phosphorylation of PECAM-1 claimed in claim 19 for the treatment of or for reducing the occurrence of cardiovascular diseases such as thrombosis, vascular occlusion or stroke, or for the treatment of or for reducing the occurrence of haemostasis disorders.

29. The activation claimed in claim 19 wherein the activation or cross-linking or phosphorylation of PECAM-1 modifies or inhibits or decreases any one selected from the group comprising: total tyrosine phosphorylation, platelet protein phosphorylation, platelet secretion from dense granules, mobilization of calcium from intracellular stores, production of inositol phosphates, and regulation of integrin-linked kinase.

30. The activation or cross-linking or phosphorylation of PECAM-1 as claimed in claim 19 for inhibiting or modifying or reducing platelet activation stimulated by ITAM or non-ITAM containing receptors or receptor agonists.

31. The activation or cross-linking or phosphorylation of PECAM-1 as claimed in claim 30 for inhibiting or reducing or modifying the activation, aggregation or secretion of platelets in response to any one selected from the group comprising; collagen, collagen related peptide (CRP), convuluxin, thrombin, ADP, thromboxane mimetics, U46619, immunoglobulin G FcγRIIA (FcγRIIA), immunoglobulin E FcεRI (FcεRI), tyrosine kinase, GPVI-mediated signalling and thrombin receptor mediated signalling.

32. A PECAM-1 activator for use in accordance with claim 19.

33. An activator as claimed in claim 32 wherein the activator is selected from the group comprising; a small molecule, an antibody, an antibody derivative, an agonist, an antagonist, a ligand, a DNA sequence, a complementary DNA sequence, an antisense DNA sequence, a probe, a protein sequence, a recombinant extracellular domain or domains of PECAM-1, a catalyst, shear, oxidative stress, FcεRI, the high affinity receptor for FcεRI, an activated form of the high affinity receptor FcγRIIA, FcγRIIA, the low affinity receptor for FcγRIIA and an activated form of the low affinity receptor FcγRIIA.

34. The activator claimed in claim 32 for the treatment of or for reducing the occurrence of cardiovascular diseases such as thrombosis, vascular occlusion or stroke, or for the treatment of or for reducing the occurrence of haemostasis disorders.

35. The activator claimed in claim 32 for the use in the manufacture of a medicament for the treatment of or for reducing the occurrence of cardiovascular diseases such as thrombosis, vascular occlusion or stroke, or for the treatment of or for reducing the occurrence of haemostasis disorders.

36. A screen for activators of PECAM-1 comprising PECAM-1, an ectodomain of PECAM-1, the cytoplasmic tail of PECAM-1, the ITIM of PECAM-1, an active site ofPECAM-1, a recombinant extracellular domain or domains of PECAM-1, or a part or derivative thereof and means for detecting activation or cross-linking or phosphorylation or tyrosine phosphorylation of PECAM-1, an ectodomain of PECAM-1, the cytoplasmic tail of PECAM-1, the IPIM of PECAM-1, an active site of PECAM-1, a recombinant extracellular domain or domains of PECAM-1 or a part or derivative thereof.
